(19) United States
(12) Patent Application Publication
(10) Pub. No.: US 2010/0198114 A1
(43) Pub. Date: Aug. 5, 2010

(54) ADJUSTING PARAMETERS OF AN APPARATUS FOR SHOCKWAVE TREATMENT

(76) Inventors: Pavel Novak, Stetten/SH (CH); Ernst H. Marlinghaus, Bottighofen/TG (CH); Manfred Schulz, Tragerwilen/TG (CH); Josef Katona, Radolfzell (DE)

Correspondence Address: WOOD, PHILLIPS, KATZ, CLARK & MORTIMER, 500 W. MADISON STREET, SUITE 3800, CHICAGO, IL, 60661, US

(21) Appl. No.: 12/698,735

(22) Filed: Feb. 2, 2010

(30) Foreign Application Priority Data
Feb. 2, 2009 (DE) 10 2009 007 129.6

(51) Int. Cl. A61B 17/225

(52) U.S. Cl. 601/4

(57) ABSTRACT

The invention relates to a method for setting a pressure application time in an apparatus (10, 44) for treating the human or animal body by mechanical shockwaves, independently of pressure and/or frequency values set, and to a respective apparatus (10, 44).

### FIELD OF THE INVENTION

The present invention relates to an apparatus for treating human or animal bodies by mechanical shockwaves.

### BACKGROUND OF THE INVENTION

Such apparatuses are known as such, in particular in the area of lithotripsy. There, body concrements, in particular stones in the body tissue, are disintegrated by focused mechanical shockwaves. Besides the production by electrical discharges in water, also apparatuses have been developed producing the mechanical shockwaves by the collision of an accelerated striking element and an impact body and coupling said shockwaves into body tissue by means of said impact body. Such apparatuses have been used also in lithotripsy by a direct contact between the impact body or a probe connected to the impact body and the stone, and in other treatments of biological body substances. In particular, the treatment of muscle diseases and of diseases in the transition region between muscles and bones are to be named.

An example for an apparatus of the type mentioned at last is shown in EP 0 991 447. There, unfocused shockwaves shall be coupled into the body tissue.

In such apparatuses, the intensity of the coupled-in shockwave can be varied by adjusting a pressure value of a pneumatic supply means. The higher the pneumatic value provided, the more the striking element is accelerated and the larger the impulse and energy transfer to the impact body is.

Moreover, many apparatuses provide for an adjustment of the repetition frequency of pneumatic pulses and thus the repetition frequency of the impacts of the striking element onto the impact body and the resulting shockwaves to be coupled-in.

Further, many apparatuses enable the exchange of impact bodies against others differing as regards geometry and/or mass.

### BRIEF SUMMARY OF THE INVENTION

The present invention is based on the technical problem to further improve such apparatuses as regards the adjustment of parameters and to provide for a respective method for adjusting.

This problem can be solved by a method for adjusting the pressure gas application time of an apparatus for treating the human or animal body by a mechanical shockwave, said apparatus comprising: a pressure gas supply device for producing gas pressure pulses repeated with a frequency, a striking element to be accelerated by a pressure gas pulse of said pressure gas supply device, an impact body to be stroken by said accelerated striking element in order to receive an impulse therefrom for producing a shockwave, an adjustment device for adjusting a pressure value and/or said frequency of said pressure gas pulses and an adjustment device for adjusting the time duration of said pressure gas pulses, in which adjustment method predetermined time duration values for respective pressure and/or frequency values are selected and set in response to pressure and frequency values set, respectively, and by an apparatus adapted correspondingly.

Preferred embodiments are defined in the dependent claims and will be explained hereunder wherein individual features generally relate both to the method category and to the apparatus category and also to preferred uses of the apparatus without any explicit differences being made therebetween hereunder.

The invention is based on the adjustment of at least one parameter, namely a pressure value or a frequency, but relates even more to situations in which both parameters are varied. Adjustment means a variation to be made by the user himself and thus not variations which are only possible in the course of maintenance. Actually, a device for varying the parameters by operating operation elements, using standard tools present or delivered with the apparatus or also by connecting another electronic device and using this device as a terminal for adjustment shall be provided. The question for which therapeutic reasons individual pressure values and/or frequency values are desired in individual cases will not be treated here. Instead, it shall be taken for granted that in certain cases and for certain indications different parameters or sets of parameters are desired, possibly even in consideration of a certain impact body selected from a given plurality.

Thus, the invention is based on the observation of the inventors that several advantages can be achieved by the time duration of the pressure pulses varying in response to other parameters. As an example, it can be observed that apparatuses of the above-described type operated pneumatically or otherwise by pressure gas work in an optimal manner only for certain parameter settings, and that for some parameter settings even functional defects can occur. Actually, this can mean that the “efficiency” for certain adjustments of pressure and/or frequency, i.e. the production of a shockwave as intense as possible for a certain value, can be optimized substantially in most cases. Basically, it can be seen as an advantage to minimize the load of the pressure gas supply device, namely to use a minimum pressure and/or minimum gas volume of the supply in order to generate a certain shockwave intensity. An adaptation can be made according to the invention by setting time duration values optimized in a certain sense for the pressure pulses. Thus, if a small-designed pressure gas supply device suffices due to such optimizing measures, this is advantageous in view of costs, weight, construction size, and energy consumption. Consequently for a certain pressure gas source, for example a compressor, the given characteristic curve for pressure and volume supply can be used in an optimal manner for maximum intensities of shockwaves achievable, which means that with given compressors relatively intense shockwaves can be produced by adjusting the time duration values. Vice versa, for a certain target area of shockwave intensities, the invention enables a use of smaller pressure gas supply devices, for example smaller compressors. However, the invention can also be useful in that a steadily proper operation can be guaranteed for pressure parameter ranges and/or frequency ranges desired by choosing adequate time duration values.

The precise correlation between the pressure values, the frequency values, and the time duration values of the pressure pulses is complex and strongly depending on the individual apparatus. In case of too short time durations (especially for a high pressure), due to the finite switching times of valves used and a pressure rise delay by flow resistances, the pressure principally achievable can not be reached any more under certain circumstances, or can only be reached in a region actually effective for the acceleration of the striking element for a time being substantially shortened. If the pressure decreases relatively early due to a short time duration, the striking element has possibly not been moved over a substantial portion of its proper acceleration distance, can be accelerated only in an insufficient manner in relation to the acceleration theoretically possible, and can even be decelerated in case of a too early collapse of the pressure front even before the actual collision, under certain circumstances.

On the other hand, problems can arise by too long pulse durations in that the accelerating pressure pulse has not yet elapsed sufficiently at the time of a back movement of the striking element into the original position. In case of a residual pressure this can lead to a new acceleration towards the impact body before a returning to the original position and thus to possibly undesired weak “after pulses” and additional weaker waves. In this respect, for further illustration reference is made to DE 20 2004 011 323 teaching to intentionally use this effect in order to produce a plurality of collisions per triggering of a pressure pulse. In many cases, this is not desired, however. This effect increases with the pressure so that too long pulse durations become more and more critical with increasing pressure.

Finally, longer pulse durations and thus usually longer valve opening times do not contribute any further to an effective use of the supply pressure provided, from a certain value on, because the striking element accelerated, from a certain velocity on, so to say flies away from the pressure gas quantity which is still flowing but not as fast as in the beginning of the pressure pulse, and thus does not depend on the pressure gas development and residual rise any more in a substantial manner.

In view of the influence of frequency it is to be mentioned that the striking element should be returned into the original position completely before triggering a new accelerating pulse. Otherwise, in the next acceleration process not the complete accelerating distance can be used. Likewise, the impact body must stand still again completely or at least substantially in order to enable a reproducible new shockwave process. Moreover, technical problems can arise if the striking element strikes before, what is considered hereunder again.

Thus, the basic correlation between the pressure values and optimal pulse duration values is substantially determined by the fact that a longer time is needed to supply sufficient air for the acceleration in case of low pressure and that, in case of a higher pressure value, shorter times may and must be chosen. In addition, the time values must be lowered in case of higher frequencies to avoid difficulties. In contrast to conventional apparatuses having fixed and predetermined time values, in case of lower frequencies and pressure values also substantially longer times can be chosen in order to improve the efficiency.

The processes summarized above do not only depend on the participating masses and the properties of the switching valves used but also in particular from the geometrical construction of the apparatus such as of the conduits through which the pressure gas flows (in particular the length and the diameter of the guiding tube as well as the geometry of the weight of the striking element, compare the embodiment). The definition of analytical correlations is practically impossible mainly due to the usually turbulent flow conditions. On the other hand, optimal or suitable time duration values for respective pressure and/or frequency values can be determined empirically without difficulty. A limited precision is absolutely acceptable, therein. Hence, the invention is not necessarily directed to an optimization of parameters in a strict sense but can already be used in a senseful manner by an approximated setting of the values. In particular, parameter sets determined stepwise can be used readily, in which context reference is made to the embodiment.

The intensity of the shockwave can be determined by measuring the travel length of the impact body for example by optical methods (interferometry), by a piezo-sensoric detection of the intensity of the impact applied by the impact body, by a measurement of pressure curves in a water tank coupled to the impact body or in another manner. The pressure values described herein normally refer to the supply side in the same sense as the time duration values of the pressure gas pulses do, i.e. to the pressures provided for example by a pressure gas bottle or a compressor without consideration of losses in conduits up to the actual location of striking element acceleration, and, respectively, to the valve switching times which result in the pressure gas pulses. Analogously, the frequencies correspond to the frequencies of the valve operation. In this sense, pressure values and frequencies of commercial apparatuses are adjustable and the time duration values can be set in the easiest manner according to the invention. It is to be understood that the invention does not depend on which exact measurement location, measurement method, or adjustment means for the respective parameter is used precisely in an individual case.

Preferably, the time duration values are set automatically and thus by the apparatus itself if a pressure value or frequency value has been changed. This is, however, not mandatory. To the contrary, the invention can already be implemented in a senseful manner by manual adjustments in response to given time duration values for certain pressure and/or frequency values according to a collection of the values in documents provided by the apparatus manufacturer or in self-made tables or from a reading on a display of the apparatus.

In any case, it is preferred to select from a table memorized in the apparatus whether this is done by a display of the value to be set manually or by an automatic adjustment.

The adjustment described in the already-mentioned utility model DE 20 2004 011 323 in view of a plurality of strikes per pulse is not preferred in the context of this invention. However, it is not excluded. For example, in case of a double strike per pulse, quite reasonable optimizations can be done by respective time duration adjustments.

The prior art just cited illustrates a preferred implementation of the apparatus wherein the striking element is not only driven by pressure gas along its movement path but also pushes gas in front of it. This gas pressurized by the striking element movement is not discharged but received in a counterpressure chamber. After completion of the acceleration the gas pressure in this counterpressure chamber can be higher than the pressure on the other side of the striking element and thus provide for a return of the striking element into the original position.

The inventors have found that in many apparatuses the pressure dependency of the time duration to be adjusted is much more pronounced than the frequency dependency. It is thus preferred to consider at least the pressure dependency. It is further preferred, however, to neglect the frequency dependency for simplifying the two-dimensional dependency of the time duration values and the thus substantially increased complexity, i.e. to concentrate on the pressure dependency. By the way, the invention also relates to apparatuses using single pulses or able to do that. Therein, adjustment ranges of for example 0.5 bar-10 bar or smaller ranges make sense. Typical frequencies can be in the range of 0 Hz-50 Hz, whereas smaller ranges can be sufficient also here. Typical suitable times can be in the range between 2 ms and 25 ms, in particular between 5 ms and 15 ms.

The invention is particularly advantageous for apparatuses constructed such that their impact body is elastically held for example by using one or preferably two elastomeric rings. It is already been mentioned that a sufficient return movement of the impact body usually moving much slower than the striking element, can be important in connection with this invention. Thus, the invention has a particularly advantageous effect in case of impact bodies that can move correspondingly, namely in case of relatively large travel amplitudes. This applies in particular to travel amplitudes in a range of more than 0.5 mm or even more than 1 mm.

The travel values are to be understood as measured with the apparatus being fixed for example by a stand and relative to the apparatus.

A further aspect connected to a special advantage of the invention is a safety catch for catching the striking element. Such safety catches have already been used to inhibit an uncontrolled speeding-out of an accidentally triggered striking element in case of removed impact body. It can be implemented for example by a narrowing of a tube portion in which the striking element is accelerated. E.g., if the striking element is reached by the next accelerating pressure pulse already in its returning movement due to incorrectly set time values and thus is re-accelerated earlier than adequate, the impact body can still be shifted out of its normal position and then the striking element can be caught in the safety catch. This can also happen in case of a too late and too slow gas discharge. The “exhaust” air is compressed by the returning striking element such that a reflection of the striking element and a second collision results before the next pressure pulse is triggered.

### BRIEF DESCRIPTION OF THE DRAWINGS

The invention will be explained in more detail along exemplary embodiments which relate to all claim categories and the individual features of which can also be important in other combinations.

FIG. 1 shows an apparatus according to the invention in longitudinal section and having a schematically illustrated pneumatic drive.

FIG. 2 shows a second embodiment wherein only a part of a hand-piece is illustrated in section.

FIG. 3 shows a variation of FIG. 2 as a third embodiment.

### DETAILED DESCRIPTION OF THE PREFERRED EMBODIMENTS

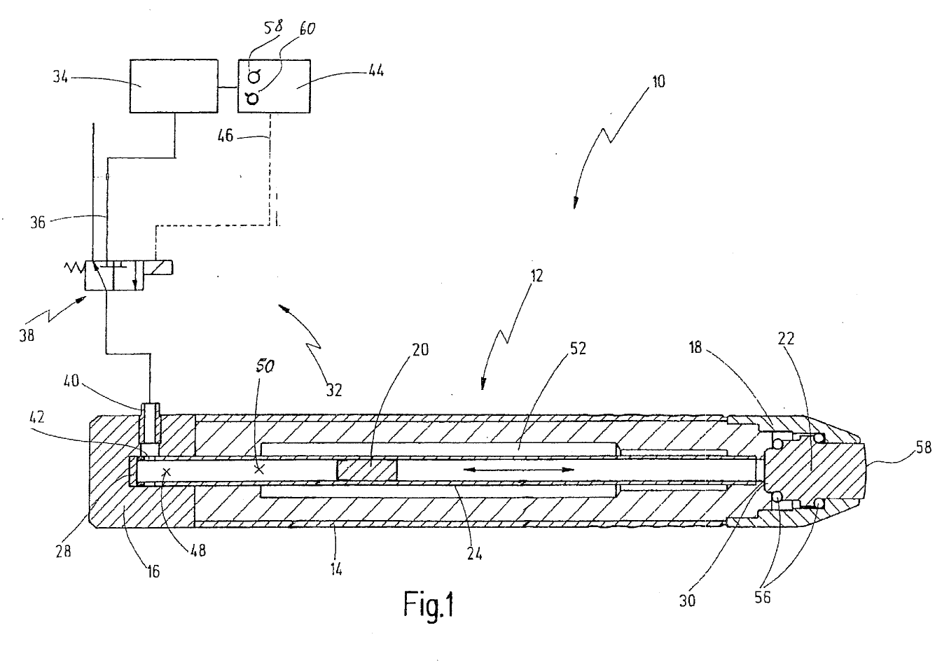

FIG. 1 shows a medical apparatus for treating the human body by mechanical shockwaves, being designated by 10, in this case adapted for a soft tissue treatment in the context of a pain treatment. The apparatus consists of a hand-piece 12 and a pneumatic pressure gas supply device 32 to be explained below in more detail. A medical doctor responsible for treatment, as an example, can grip the hand-piece 12 and position the right end in FIG. 1 onto a suitable skin portion wherein the hand-piece 12 is approximately orthogonal to the skin.

A casing 14 has a proximal terminal cap 16 and a distal terminal cap 18 being removable respectively. A guiding tube 24 is held in the casing and is arranged axially and concentrically. A striking element 20 is guided in the guiding tube, the movement path of which striking element along the interior of the guiding tube 24 being limited on the right side by an impact body 22, namely by its proximal side 30. This constitutes a distal abutment stop for the striking element 20 wherein the proximal abutment stop of the striking element 20 is designated by 28 and is a simple closure of the guiding tube 24. This closure is magnetic so that the striking element 20 can be fixed along by certain holding force. Typically, the length of the guiding tube 24 is about 5 cm-20 cm wherein the embodiment shown here approximately in scale has a guiding tube length of 17.4 cm.

The pneumatic drive 32 implements the pressure gas supply device and comprises a common pneumatic compressor 34 (or a pressure gas bottle), wherein the compressor 34 has a typical operation range up to about 10 bar. A pressure gas terminal 40 of the hand-piece 12 is supplied via a pressure conduit 36 and a switching valve 38, which terminal 40 communicates with the guiding tube 24 via the opening 42 therein. The switching valve 38 can be a magnetic valve. A control 44 is connected thereto via a control line 46 being illustrated by a hatched line. The control 44 can be implemented as a structural unit with the compressor 34 and thus constitute a basic device for supplying the hand-piece 12 wherein the switching valve 38 is advantageously arranged at the latter. This has the advantage that the volume to be filled by the pressure pulse is small. Thus, stronger and faster pulses can be realized. Correspondingly, the control 44 and the compressor 34 in FIG. 1 are connected by a line. The basic device and the hand-piece 12 are then connected via a pneumatic conduit 36 and the control line 46 combined in a supply line.

Two adjustment buttons 58 and 60 are provided on the control 44 whereby the maximum supply pressure provided by the conduit 36 and the operation frequency of the switching valve 38 can be set. The adjustment button 58 serves for adjusting the pressure valve of the compressor 34 by means of the line shown between the control 44 and the compressor 34. (If the control 44 would also be integrated with the hand-piece 12 in a structural sense, the adjustment button 58 could be provided on the compressor 34 itself or a respective control line could connect the hand-piece 12 and the compressor 34.) The control 44 is adapted to control the switching valve 38 with a frequency set at the adjustment button 60 in a range of 0 Hz-50 Hz, and sets respective opening times of the valve, therein, which are selected automatically in response to the pressure value set at the adjustment button 58. Therein, the control 44 follows a look-up table memorized and comprising for example the following values:

| | Pressure value (bar) | | | | | | | | |
|---|---|---|---|---|---|---|---|---|---|
| | up to 1.0 | 1.0-1.2 | 1.2-1.6 | 1.6-1.8 | 1.8-2.2 | 2.2-2.8 | 2.8-3.4 | 3.4-4.2 | from 4.2 |
| Time duration | 14 | 13 | 12 | 11 | 10 | 9 | 8 | 7 | 6 |
| (ms) | | | | | | | | | |

This embodiment refrains from a variation of the switching time in dependency from the frequency in favour of a simple construction. In a more complex embodiment, the above table would be a two-dimensional matrix so that the switching times would depend on the pressure value set and the frequency value set within certain respective ranges. A basic rule for many cases is that the pressure pulses need to become shorter with increasing pressure and increasing frequency.

Starting from a non-operating condition of the apparatus 10, i.e. at the start of operation, the closed switching valve 38 is opened by the control 44. The condition shown in FIG. 1 in which the guiding tube 24 is connected to the exterior atmosphere is then changed into a condition shown by the right square of the valve symbol wherein the supply pressure is applied to the guiding tube 24 via the terminal 40. Therein, the striking element 20 is in its original position, first, designated by 48 in FIG. 1. The rising pressure accelerates the striking element 20 towards the impact body and is decreased even before the collision by a back-switching of the switching valve 38 and thus by a ventilation of the volume “behind” the striking element 20 in the guiding tube 24, however. The striking element 20 hits the impact body 22 directly, the distal (somewhat convex) terminal surface 58 of which is positioned on the skin of the patient and transfers a mechanical shockwave into the body. Therein, the impact body 22 is subjected to an axial travel due to its elastic suspension in the two elastomer O-rings 56.

Directly after the collision, the striking element 20 is moved backwards. This is assisted by a counterpressure chamber 52 being connected to the guiding tube 24, namely its distal end short before the proximal side 30 of the impact body 22, in a manner not shown in detail here. A counterpressure returning the striking element 20 after the collision up to the proximal stop, namely the magnetic terminal piece 28, results from the air shift due to the movement of the striking element 20. This process shall not be inhibited substantially by a remaining residual pressure due to a too late switching of switching valve 38 which would for example result in that only the position 50 is reached instead of the optimal position 48. On the other hand, for a given supply pressure set, a respective maximal intensity of the shockwave produced shall be reached so that certain treatment results can be achieved by comparatively low pressure values and a low gas consumption, respectively. Thus, the pressure gas supply 32 and namely the compressor 34 can be designed in a small dimension.

After a certain time, the switching valve 38 is switched again so that a new trigger process results. This certain time and the on-time of the switching valve 38 make up the inverse value of the frequency set, together. The control 44 is designed such that even for higher frequencies that can be set no complications due to too long switching times can result. Principally, this could mean for relatively high frequencies that the collision intensity becomes frequency-dependent as well for identical pressure values set because in view of the frequency the switching times must be shortened. In case of necessity, this can be solved by an automatic increase of the pressure so that the adjustment of the pressure is actually an adjustment of the intensity and that the collision intensity remains independent from the frequency, however.

Typical collision velocities of the striking element are in the range of 5 m/s-60 m/s.

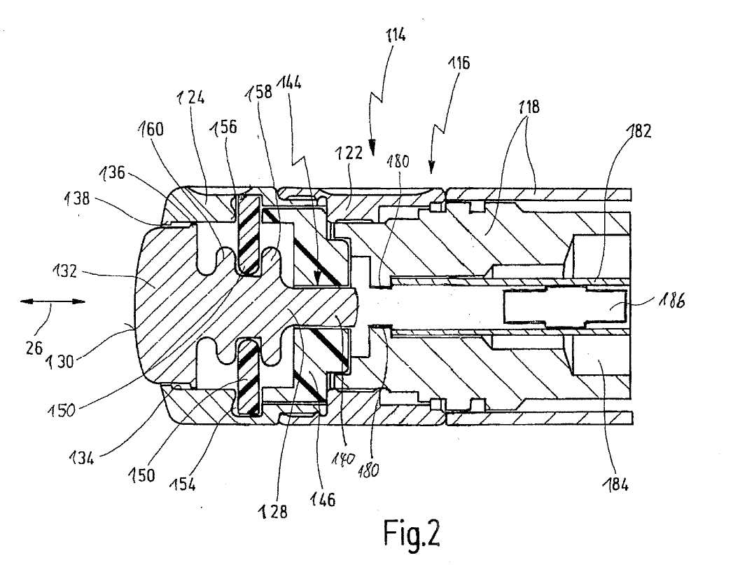

The second embodiment in FIG. 2 is shown only as a portion and in section, namely as an alternative implementation of the distal part of the hand-piece.

The hand-piece has an instrument top 114 wherein a proximal casing portion 118 serves for a removable connection to a casing of the hand-piece. Two sleeves 122 and 124 are screw-fixed to the casing portion 118 and to the other sleeve, respectively, and thus removable. The impact body is numerated by 128, here, and has a shape differing substantially from the first embodiment but being rotationally symmetric again with regard to the axis of the hand-piece. Further, the surface 130 to be positioned onto the skin corresponds substantially to the distal surface 58 of the first embodiment. It is formed on a head 132 of the impact body 128 being held in an opening 134 of the casing 116 in a manner enabling a free axial and longitudinal movement. Therein, the impact body is secured for the case of a crack by shoulders 136 and 138 of the impact body and at the opening 134, respectively, and is supported by means of a shaft 140 in a PEEK or PTFE sliding bearing 144 in a bush 146 and, further, in an elastomer flat ring 150. The elastomer ring lies against a shoulder 156 only, in a pocket 154 as regards the axial direction and is held on the shaft 140 by ring flanges 158, 160. A suitable material for the flat ring 150 can be silicone rubber or nitrile rubber (NBR). In any case, it serves for the elastic suspension of axial reciprocating movements of the impact body 128.

Therein, the complete instrument top 114 is not only removable, but also decomposable into the elements described so that they can be exchanged individually.

The ballistic mechanism for producing the shockwaves in the and by means of the impact body 128 corresponds to the explanations of the first embodiment. In particular, the guiding tube is numerated by 182, here, the counterpressure chamber by 184 and the striking element by 186 which has a shape having tapered ends to which reference will be made hereunder. In this embodiment, beside the more complex construction a softer suspension of the impact body 128 in the axial direction is provided so that the typical travel distances of the movement of the impact body 128 are substantially larger. They are typically above 1 mm. The impact body 128 can be made of metal, such as steel or stainless steel, preferably hardened, of hard synthetics or also of suitable ceramics, such as the impact body 22 of the first embodiment.

Since the impact body 128 travels along a larger distance and in a softer suspension, its overall movement until the return into the original position shown is longer than in the first embodiment in total. This increases the risk that the impact body 128 has not yet returned completely as soon as the striking element 186 approaches again. Thus, the striking element 186 could penetrate into the distal tubular opening and becomes stuck there by its radially thicker middle portion in an only sketched and small inner diameter reduction 180 directly adjacent to the distal end of the tube 182. This security catch 180 is provided for security reasons. This security catch 180 is provided for security reasons in order to inhibit a speeding-out of the striking element in case of the instrument top 114 being demounted. An impact of the striking element into the security catch 180 would cause a damage so that a condition of operation connected with a double stroke of the striking element 186 due to only one pressure pulse—being undesired at least in the context of this embodiment—would lead to substantial problems in this sense. Thus, special care must be taken of that the striking element 186 will not be reaccelerated once more towards impact body 128 during its return movement by a residual pressure gas “cushion”. Principally, a comparable problem could arise when setting particularly high operation frequencies, i.e. if the next regular pressure gas pulse arrives correspondingly early. This, however, would be a problem of a too high operation frequency in relation to the impact body dynamics and is more a theoretical limit.

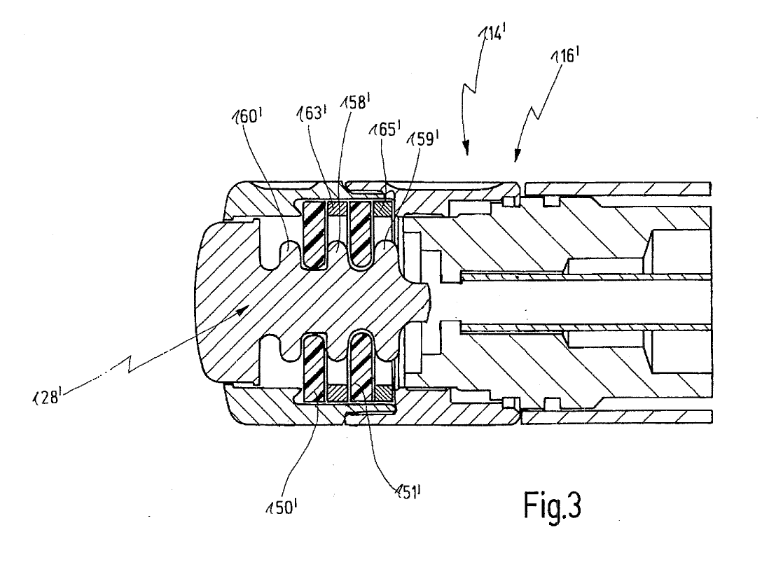

FIG. 3 shows a variation of FIG. 2 wherein the respective reference numerals have an additional dash. In place of the above-mentioned sliding bearing 144, here a double support is already given by the elastic suspension due to a second elastic flat ring 151′. Correspondingly, an additional ring flange 159′ is provided. A spacer ring 163′ is provided between flat rings 150′ and 151′, wherein the arrangement is fixed by a clamp ring 165′.

Apart from these differences, the structure and operation of the variation shown in FIG. 3 are substantially the same as the structure and operation of the apparatus shown in FIG. 2, as described above.

What is claimed is:

1. A method for adjusting the pressure gas application time of an apparatus for treating the human or animal body by a mechanical shockwave,
said apparatus comprising:
a pressure gas supply device for producing gas pressure pulses repeated with a frequency,
a striking element to be accelerated by a pressure gas pulse of said pressure gas supply device,
an impact body to be stroken by said accelerated striking element in order to receive an impulse therefrom for producing a shockwave,
an adjustment device for adjusting a pressure value and/or said frequency of said pressure gas pulses and an adjustment device for adjusting the time duration of said pressure gas pulses,
in which adjustment method predetermined time duration values for respective pressure and/or frequency values are selected and set in response to pressure and frequency values set, respectively.

2. The method of claim 1 wherein said selection and setting of the time duration values is done automatically by said apparatus.

3. The method of claim 1 wherein said selection of said time duration values is performed from a table memorized within said apparatus.

4. The method of claim 1 wherein said time duration values are set such that only one respective collision of said striking element onto said impact body is allowed per pressure gas pulse.

5. The method of claim 1 wherein said apparatus is constructed such that said striking element displaces air in front thereof along a path of its acceleration and presses said air into a counterpressure chamber in which a counterpressure is caused, consequently, for returning said striking element along said path.

6. The method of claim 1 wherein said apparatus comprises a device for adjusting pressure values of said pressure gas pulses and wherein said time duration values are selected and set in response to said pressure values set.

7. The method of claim 6 wherein said time duration values are selected and set exclusively in response to said pressure values set.

8. The method of claim 1 wherein said impact body is suspended elastically so that it can be displaced by an impulse transfer from said striking element.

9. The method of claim 8 wherein said apparatus is adapted such that values of said displacement in a range of 0.5 mm to 5 mm result.

10. The method of claim 8 wherein said suspension comprises at least one elastomer ring.

11. The method of claim 1 wherein said apparatus comprises a security catch for catching said striking element in case of triggering said acceleration thereof while said impact body is dismounted

12. The method of claim 11 wherein said security catch comprises a tapering at an end to said impact body's side of a tube portion for accelerating said striking element.

13. An apparatus for treating the human or animal body by a mechanical shockwave, comprising:
a pressure gas supply device for producing gas pressure pulses repeated with a frequency,
a striking element to be accelerated by a pressure gas pulse of said pressure gas supply device,
an impact body to be stroken by said accelerated striking element in order to receive an impulse therefrom for producing a shockwave,
an adjustment device for adjusting a pressure value and/or said frequency of said pressure gas pulses and an adjustment device for adjusting the time duration of said pressure gas pulses,

14. A method of treating a soft tissue of the human or animal body by a mechanical shockwave comprising use of the apparatus of claim 13.
